(19) United States
(12) Patent Application Publication
(10) Pub. No.: US 2009/0198124 A1
(43) Pub. Date: Aug. 6, 2009

(54) WORKFLOW TO ENHANCE A TRANSJUGULAR INTRAHEPATIC PORTOSYSTEMIC SHUNT PROCEDURE

(76) Inventors: Ralf Adamus, Nurnberg (DE); Marcus Pfister, Bubenreuth (DE)

Correspondence Address: SCHIFF HARDIN, LLP;PATENT DEPARTMENT, 6600 SEARS TOWER, CHICAGO, IL, 60606-6473, US

(21) Appl. No.: 12/023,906

(22) Filed: Jan. 31, 2008

(51) Int. Cl. A61B 6/00

(52) U.S. Cl. 600/424

(57) ABSTRACT

A method and appertaining system are provided for performing an image-assisted subject procedure. The method comprises obtaining at least a first and a second 2D projected image of a 3D subject from a first angle and a second angle. A user selects at least a first point and a second point in each of the first and second 2D images, and the system calculates 3D positions of the first point and the second point based on the selected points in the 3D images and the first and second angles. The, a 3D line is calculated between the first and second points. A further projected 2D image is created that includes the calculated 3D position of at least one of: a) one of the points; and b) the line. The subject procedure is then subsequently performed utilizing the further projected 2D image as a guide.

### CROSS-REFERENCE TO RELATED APPLICATIONS

The present application is related to the subject matter in the following pending U.S. patent applications, all of which are herein incorporated by reference: U.S. Ser. No. 11/298,772, filed Dec. 9, 2005; U.S. Ser. No. 11/266,886, filed Nov. 4, 2005; U.S. Ser. No. 11/540,621, filed Dec. 9, 2005; U.S. Ser. No. 11/544,846, filed Oct. 5, 2006; and U.S. Ser. No. 11/900,261, filed Sep. 11, 2007.

### BACKGROUND

The present invention is related to angiographic systems and techniques that are used to image and register the position of instruments within the body during medical procedures, particularly a transjugular intrahepatic proto-systemic shunt (TIPS) procedure, which is a challenging interventional abdominal procedure performed on an angiographic system (such as the Siemens AXIOM Artis™).

During this procedure, the radiologist puts a long, curved needle through a catheter placed in the jugular vein (entering in at the patient's neck) through the liver vein (or systemic vein). This needle will then be pushed from the patient's liver vein, (or systemic vein) into the portal vein, which lies close to it. Once the needle has been passed between liver vein and portal vein, a stent will be placed to keep this channel open.

The reason for this procedure is to address a (partial) blocking of the portal vein system due to specific liver diseases like liver cirrhosis. This blocking causes the blood pressure in the portal vein to rise. Because of this, the patient may have developed dangerous varicose veins inside the abdomen. After the TIPS, blood will flow from the high-pressure portal vein into the low-pressure liver (or systemic) vein. The high pressure in the portal vein will then consequently be reduced, back towards normal.

The problem with this procedure is that the radiologist must perform the puncture more or less blindly, since he can not see the portal veins. Also the portal system has to be hit inside of the liver tissue, otherwise the patient has a high risk of bleeding.

It is known in the art to provide for calibration of angio systems. It is also known to provide for a registration of 2D and 3D images, from, e.g., G. P. Penney. Registration of Tomographic Images to X-ray Projections for Use in Image Guided Interventions. Phd. Thesis, University College London, CISG, Division of Radiological Sciences, Guy's Hospital, King's College London, London SE1 9RT England, 2000. (Pages 36-58).

It is also known to provide such a registration of 2D and 3D images in various medical applications, especially interventions—see, e.g., U.S. Ser. No. 11/544,846, and German Patent Application DE 102005012985 (translation in Appendix A).

It is desirable to keep an instrument (e.g., a needle) on a predefined path in 3D as described in DE 102005012985, although this path is chosen in a different way as described below.

The idea to localize structures (e.g., the tip of a device or an anatomical structure) by marking them in two 2D images from different angles and computing a point in 3D by triangulation is also previously described in U.S. Ser. No. 11/900,261.

What can be done today to overcome this problem is to visualize the portal system (i.e., the puncture target), mainly by two mechanisms: 1) an additional catheter is placed up to the liver arteries to be able to inject contrast agent which then flows to the portal system; and 2) the liver veins (where the first catheter is placed) are blocked by a balloon and CO2 is injected under high pressure. The gas then diffuses through the liver and highlights the portal system, giving a “negative contrast” image.

Previously, the skilled radiologist would take two of those images under different angles and, with lengthy estimations of certain angles in those images, gain some information relevant to determining the puncture direction.

It is also possible to register a 3D volume showing the portal system (e.g. a CT or MR) and overlaying the 3D portal system to the live fluoro image, as described in U.S. Ser. No. 11/544,846.

### SUMMARY

It would be very beneficial to re-project the puncture path into 3D space (and, if available, into a registered 3D volume showing additional details) where it can be viewed from different angles. Thus, according to various embodiments of the invention, the process of automating this procedure of estimating the puncture direction is desirable. After that, a calculation of the estimated puncture line in 3D is performed, and this estimated puncture line is overlaid in the fluoro image from any angulation so that the radiologist can compare planned and actual needle progress.

In more detail, a method is provided for performing an image-assisted subject procedure, comprising: obtaining at least a first and a second 2D projected image of a 3D subject from a first angle and a second angle; selecting at least a first point and a second point in each of the first and second 2D images; calculating 3D positions of the first point and the second point based on the selected points in the 3D images and the first and second angles; calculating a 3D line between the first and second points; creating a further projected 2D image that includes the calculated 3D position of at least one of: a) one of the points; and b) the line; and performing the subject procedure utilizing the further projected 2D image as a guide.

An appertaining system is provided for performing an image-assisted subject procedure, comprising: an imaging system comprising an image acquisition device for acquiring an image of a 3D subject; a processor used to process acquired images; a memory for storing acquired images and processed images; a mechanism that can be used to orient the imaging system to capture a first and a second 2D image of the 3D subject from a first angle and a second angle; a display for providing the first and second 2D images to a user; a selection mechanism via which the user can select a first point and a second point on both the first and second 2D images; a software module that calculates 3D positions of the first point and the second point based on the selected points in the 3D images and the first and second angles and calculates a 3D line between the first and second points; and a display, which is one of the display and a further display upon which a further projected 2D image is provided that includes the calculated 3D position of at least one of: a) one of the points; and b) the line.

Advantageously, the system and method allows the medical professional to define a path, e.g., for a puncture, by 2D images. This path is displayed in live fluoro images (to guide the needle) but can also be re-projected in 3D to ensure for a better visualization and assessment of the planned puncture path.

### DESCRIPTION OF THE DRAWINGS

The invention is described with reference to various embodiments as illustrated in the drawings and described in more detail below.

FIG. 1 is a flowchart illustrating a preferred embodiment of the inventive method;

FIGS. 2A, B are isometric pictorial illustrations showing a C-arm imaging a subject at different angles;

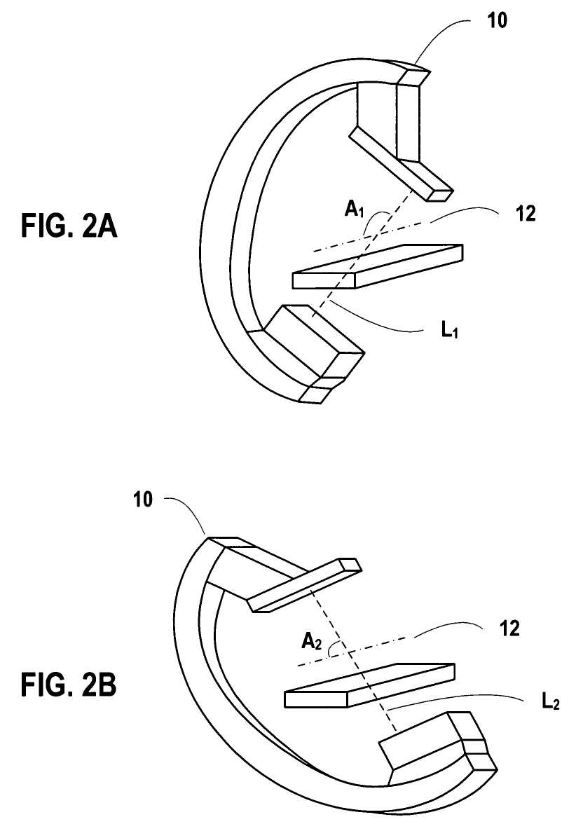

FIGS. 3A, B are pictorial illustrations showing the 3D localization of a target which can be seen in the images taken from different angles;

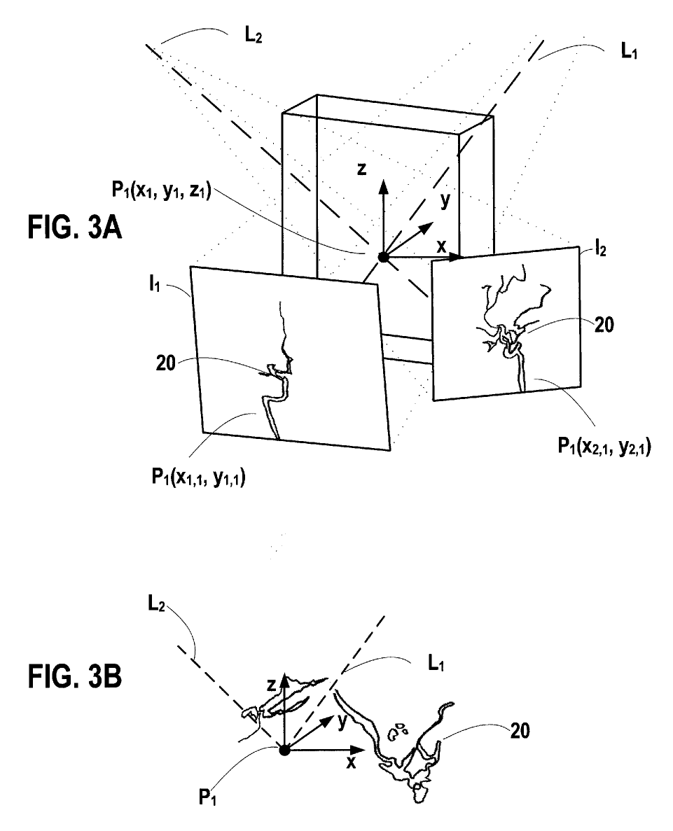

FIGS. 4A, B are pictorial illustrations of the images from different angles used for the TIPS puncture, showing the marked points;

FIG. 5 is a pictorial illustration showing the 3D determination of a line from two imaged points; and

FIG. 6 is a pictorial illustration showing the computed points and line projected into different 2D images.

### DETAILED DESCRIPTION OF THE PREFERRED EMBODIMENTS

In a preferred embodiment of the invention, a C-arm 10 angio system, such as the Siemens AXIOM Artis dTA™, is, as a preliminary matter, calibrated such that a full knowledge (i.e., the exact geometry of the system) exists as to how a point in 3D space imaged with the system projects to the detector. This knowledge is usually gained using a special phantom with balls having a known geometry. This can be either a conventional C-Arm system or a C-Arm system mounted on a robotic stand.

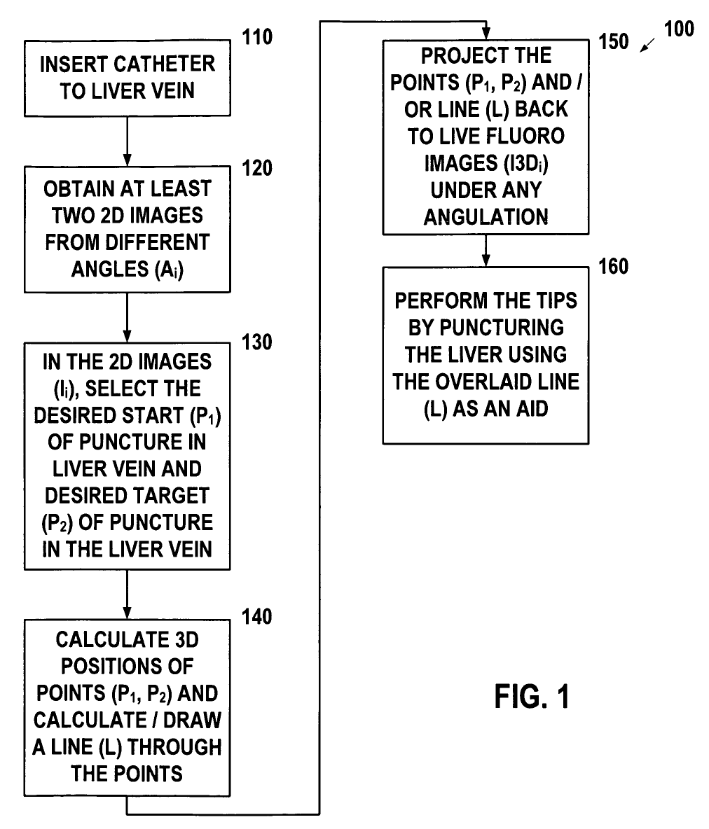

A software system is capable of back-projecting a point in 3D (see FIG. 1) by: 1) making use of this calibration; 2) having two 2D images I₁, I₂ from different angles A₁, A₂ of the structure to back-project; and 3) utilizing information as to where this structure is in the 2D image (e.g., by manually marking the structure). One desirable feature is to provide a 3D dataset of the liver via a preoperative procedure (by way of, e.g., a CT, MR, or other imaging procedure), or via an intraoperative procedure (by way of, e.g., DynaCT, 3D Angio) which is registered to the C-Arm 10.

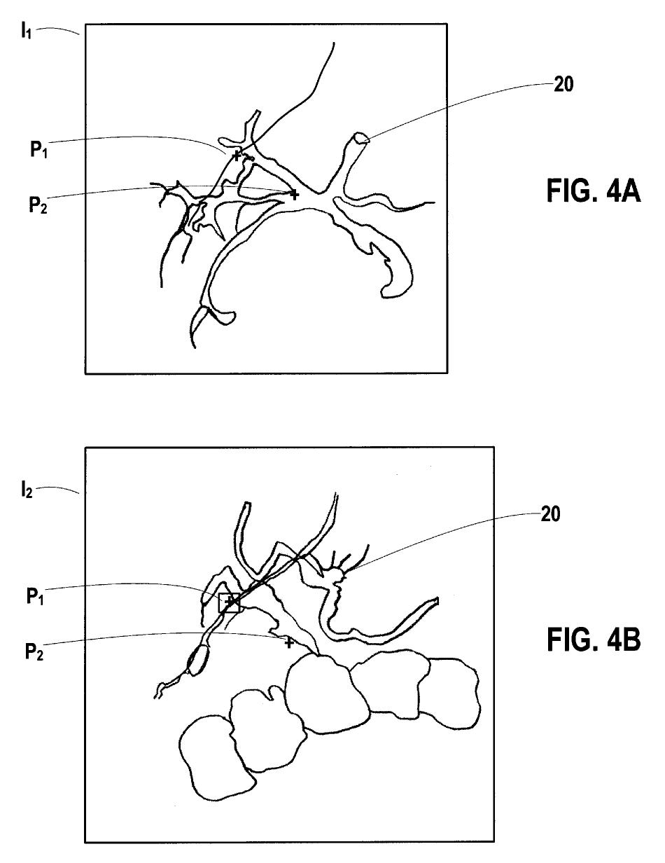

According to an embodiment of the method 100 for a work flow illustrated in FIG. 1, in Step 1, a medical professional inserts a catheter up to the liver vein 20 (FIGS. 4A, B) 110. In Step 2, the medical professional then gets at least two 2D images I₁, I₂ (FIGS. 4A, B) 120 by performing the following steps: driving the C-Arm 10 to a first proper angulation A₁; and getting an image I₁ from that angle A₁ (e.g., a CO2 angiographic image), then driving the C-Arm 10 to a second proper angulation A₂ by rotating it about the C-arm axis 12; and getting an image I₂ from that angle A₂. The imaging is performed about imaging lines L₁ and L₂ respectively.

In Step 3, the medical professional (manually) localizes 130 in the images I_{1, 2} (e.g., by selecting on them with a pointing device) at least a desired start position P₁(_{X1,1, Y1,1}) (in the first image I₁) and P₁(_{X2,1, Y2,1}) (in the second image I₂) of the puncture in the liver vein 20, and a desired target P₂(_{X1,2, Y1,2}) (in the first image I₁) and P₂(_{X2,2, Y2,2}) (in the second image I₂) of the puncture in the liver vein 20.

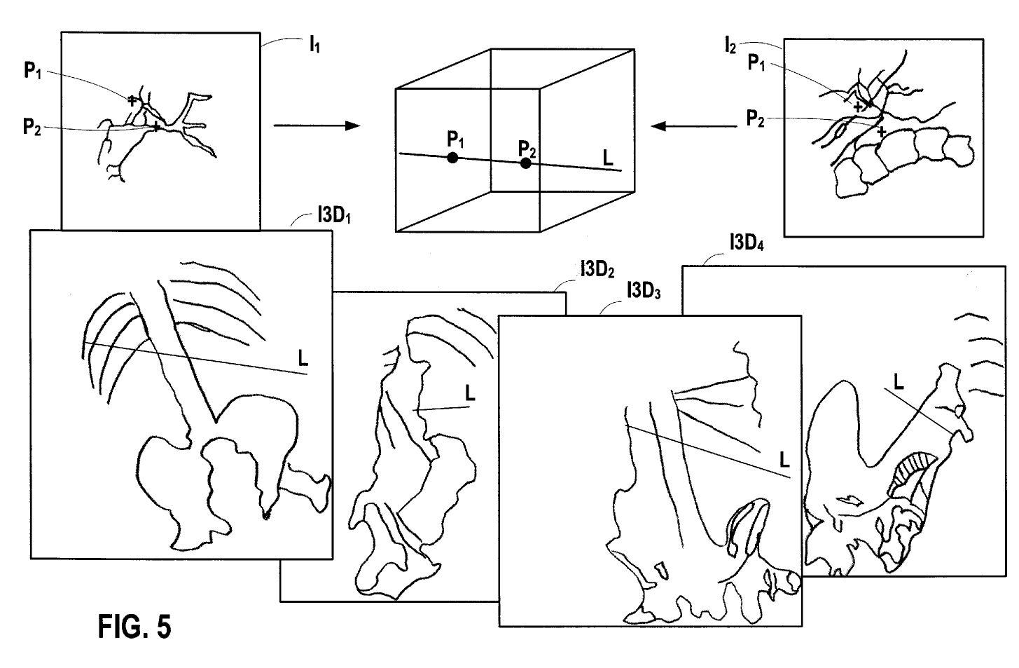

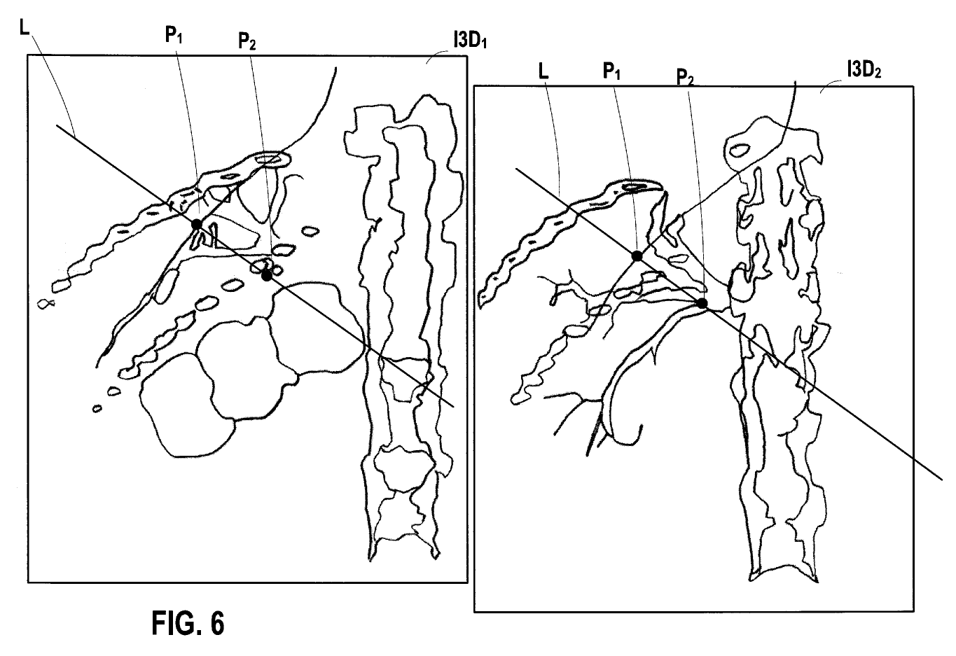

Referring to FIGS. 5 and 6, in Step 4, the system calculates 140 the 3D positions of the two points P₁(x₁,y₁,z₁), P₂(x₂,y₂,z₂) via triangulation and draws a line L through the two points P₁, P₂. In Step 5, the system projects points P₁, P₂ and/or the line L 150 back to the live fluoro images I3Dᵢ under any angulation. Finally, in Step 6, the medical professional performs the TIPS 160 by puncturing the liver using the overlaid line L as an aid.

In an alternate embodiment of the method work flow, a 3D volume V can be registered to the C-Arm (e.g., in a CT, MR, DynaCT or 3D angio system)—the volume should utilize the same coordinate system as the C-arm or be readily transposable). The puncture points P₁, P₂ and/or line L are then back-projected to that volume V. An advantage of this approach is that it can be seen if the planned path hits important anatomical structures.

The tip of the needle can be tracked in 3D, e.g., by the techniques described in U.S. Ser. No. 11/900,261, or with a position sensor, and the deviation of the tip from the planned path can be calculated and displayed. Also the tip of the needle can be displayed in 3D or within a registered 3D volume. The tip of the needle can be tracked in (one or more) 2D images, e.g., by the techniques described in [4], with a position sensor, or automatically using a proper image processing algorithm.

In Step 2, different types of images can be imagined, such as native X-ray images, iodine contrast enhanced images from the liver and portal system (e.g., by inserting the catheter in the liver artery) as long as the structures to localized (especially start and end of the puncture line) can be seen in them.

In Step 3, an arbitrary number of intermediate points can be marked and displayed. This could lead to a curved puncture line. In this step, as a puncture start, also the tip of the needle can be marked as a starting point.

Thus, according to these embodiments, a calculation and display of a puncture path is determined in 3D, and is potentially projected to a registered 3D volume and/or back-projected to the 2D live fluoro images by marking a start and end of the puncture path, e.g., by at least two images from different angulations (such as via CO2 angiographic images). This system and workflow can be used for any application which can make use of this feature of guiding a device through a volume (medical or not) by the described features.

The system or systems may be implemented on any general purpose computer or computers and the components may be implemented as dedicated applications or in client-server architectures, including a web-based architecture. Any of the computers may comprise a processor, a memory for storing program data and executing it, a permanent storage such as a disk drive, a communications port for handling communications with external devices, and user interface devices, including a display, keyboard, mouse, etc. When software modules are involved, these software modules may be stored as program instructions executable on the processor on media such as tape, CD-ROM, etc., where this media can be read by the computer, stored in the memory, and executed by the processor.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The present invention may be described in terms of functional block components and various processing steps. Such functional blocks may be realized by any number of hardware and/or software components configured to perform the specified functions. For example, the present invention may employ various integrated circuit components, e.g., memory elements, processing elements, logic elements, look-up tables, and the like, which may carry out a variety of functions under the control of one or more microprocessors or other control devices. Similarly, where the elements of the present invention are implemented using software programming or software elements the invention may be implemented with any programming or scripting language such as C, C++, C#, Java, assembler, or the like, with the various algorithms being implemented with any combination of data structures, objects, processes, routines or other programming elements. Furthermore, the present invention could employ any number of conventional techniques for electronics configuration, signal processing and/or control, data processing and the like. The word mechanism is used broadly and is not limited to mechanical or physical embodiments, but can include software routines in conjunction with processors, etc.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

What is claimed is:

1. A method for performing an image-assisted subject procedure, comprising:
obtaining at least a first and a second 2D projected image of a 3D subject from a first angle and a second angle;
selecting at least a first point and a second point in each of the first and second 2D images;
calculating 3D positions of the first point and the second point based on the selected points in the 3D images and the first and second angles;
calculating a 3D line between the first and second points;
creating a further projected 2D image that includes the calculated 3D position of at least one of: a) one of the points; and b) the line; and
performing the subject procedure utilizing the further projected 2D image as a guide.

2. The method according to claim 1, further comprising a preliminary step of calibrating an imaging system used to obtain the images.

3. The method according to claim 1, wherein the step of selecting comprises manually selecting the points on an image display with a pointing device.

4. The method according to claim 1, further comprising providing a 3D dataset of a subject feature via at least one of: a) a preoperative imaging selected from the group consisting of CT and MR; and b) an intra operative procedure selected from the group consisting of a Dyna CT and 3D Angio.

5. The method according to claim 4, wherein the 3D dataset is registered in a C-arm system used for the imaging.

6. The method according to claim 4, wherein the further projected 2D image includes the subject feature.

7. The method according to claim 1, further comprising:
selecting a third point in each of the first and second 2D images;
calculating a 3D position of the third point based on the selected point in the 3D images and the first and second angles;
calculating a second 3D line between the second and third points; and
utilizing the 3D line and the second 3D line to define a path.

8. The method according to claim 1, further comprising determining if the line intersects with an important structure of the subject.

9. The method according to claim 1, further comprising tracking a position of a medical device within the subject during the subject procedure.

10. The method according to claim 9, wherein the medical device is a catheter or a needle.

11. The method according to claim 9, further comprising calculating a deviation of the position of the medical device from the line and providing the deviation as an output.

12. The method according to claim 9, further comprising:
providing a 3D dataset of a subject feature; and
tracking the position of the medical device within the 3D dataset of the subject feature.

13. The method according to claim 12, wherein the position is tracked with a position sensor.

14. The method according to claim 1, wherein the subject procedure is a TIPS procedure that utilizes a C-arm angio system.

15. The method according to claim 14, further comprising puncturing a liver of the subject with a needle.

16. A system for performing an image-assisted subject procedure, comprising:
an imaging system comprising an image acquisition device for acquiring an image of a 3D subject;
a processor used to process acquired images;
a memory for storing acquired images and processed images;
a mechanism that can be used to orient the imaging system to capture a first and a second 2D image of the 3D subject from a first angle and a second angle;
a display for providing the first and second 2D images to a user;
a selection mechanism via which the user can select a first point and a second point on both the first and second 2D images;
a software module that calculates 3D positions of the first point and the second point based on the selected points in the 3D images and the first and second angles and calculates a 3D line between the first and second points; and
a display, which is one of the display and a further display upon which a further projected 2D image is provided that includes the calculated 3D position of at least one of: a) one of the points; and b) the line.
